(19) United States
(12) Patent Application Publication
(10) Pub. No.: US 2008/0254088 A1
(43) Pub. Date: Oct. 16, 2008

(54) SHAPED BODIES BASED ON A CROSS-LINKED, GELATINOUS MATERIAL, METHOD FOR PRODUCING SUCH BODIES AND USE OF THE BODIES

(75) Inventors: Michael Ahlers, Eberbach (DE); Melanie Rupp, Eberbach (DE)

Correspondence Address: LEYDIG VOIT & MAYER, LTD, 700 THIRTEENTH ST. NW, SUITE 300, WASHINGTON, DC, 20005-3960, US

(73) Assignee: GELITA AG, Eberbach (DE)

(21) Appl. No.: 12/120,320

(22) Filed: May 14, 2008

Related U.S. Application Data
(63) Continuation of application No. PCT/EP2006/010973, filed on Nov. 16, 2006.

(30) Foreign Application Priority Data
Nov. 17, 2005 (DE) 10 2005 054 938.1

(51) Int. Cl. A61F 2/00; A61K 47/42; A61K 35/12

(52) U.S. Cl. 424/422; 514/774; 424/93.7

(57) ABSTRACT

Shaped bodies based on gelatin which have both high mechanical strength and also sufficient flexibility are provided, the shaped body comprising a cross-linked, gelatinous material which comprises gelatin and a plasticizer, the shaped body being stretched so that the gelatin molecules are oriented at least in part in a preferred direction.

### CROSS-REFERENCE TO RELATED APPLICATIONS

This patent application is a continuation of PCT Application No. PCT/EP2006/010973, filed Nov. 16, 2006, which claims priority of German patent Application No. 10 2005 054 938.1, filed Nov. 17, 2005, which are each incorporated by reference.

### BACKGROUND OF THE INVENTION

The present invention relates to shaped bodies based on a cross-linked, gelatinous material. The invention also relates to a method for producing bodies of this kind.

The invention furthermore relates to use of these bodies in the medical field, in particular for producing implants.

Shaped bodies of resorbable materials are used in different fields in medicine, on the one hand to cover over wounds or internal or external bleeding, as well as to produce implants, which fulfil a carrier, protective or guide function. Especial importance relates to so-called tissue implants in which constructions of a resorbable material and living cells are involved (tissue engineering). These are use for treating damaged tissues and organs, in particular for regeneration of skin or cartilage.

Materials of this kind must provide a number of features in order for them to be able to be used successfully in the medical field. On the one hand, they must have sufficient strength in order to facilitate their being handled without suffering damage and to protect growing cells in the body from mechanical stress. At the same time, the material should however be flexible enough to adapt itself to the shape of the body location to be treated.

It has been found that gelatin is well suited as a base material in order to fulfil the requirements identified. Gelatin can be fully resorbed by the body and has in this regard an advantage compared with other materials such as for example chitosan, alginate, agarose and hyaluronic acid. In contrast to the related material collagen, gelatin of high purity and reproducible composition is available and is free from immunogenic telopeptides, which can cause defensive reactions by the body.

In order to achieve sufficiently long stability of the shaped body under physiological conditions, the gelatin must as a rule be cross-linked, chemically or enzymatically. The residue-free resorbability is not affected by this, but the resorption time may in each case be individually set by the degree of cross-linking.

A method for producing shaped bodies of this kind based on cross-linked gelatin is described in the German patent application with the File No. DE 10 2004 024 635.

For certain uses, a very high strength is however desirable for the shaped body and this cannot be achieved solely by raising the degree of cross-linking.

It has been described that the tear strength of gelatin films can be increased by stretching the films (Bigi at al. (1998) Biomaterials 19, 2335-2340). However, the films described in this publication, which are cross-linked using glutaraldehyde after stretching, have an ultimate elongation of less than 11%. Films of this kind do not provide the flexibility which is desirable for use in the medical field.

### BRIEF SUMMARY OF THE INVENTION

It is an object of the present invention to provide shaped bodies based on gelatin which have both high mechanical strength and also sufficient flexibility.

This object is met according to the invention by a shaped body based on a cross-linked, gelatinous material, the shaped body being stretched so that the gelatin molecules are oriented at least in part in a preferred direction, and the material comprising a plasticizer.

Surprisingly, shaped bodies based on gelatin, which on the one hand contain a plasticizer and on the other hand are cross-linked, can be stretched especially well. By virtue of the stretching, the mechanical properties, in particular tear strength and ultimate elongation, are markedly improved.

### DETAILED DESCRIPTION OF THE INVENTION

The gelatinous material, on the basis of which the shaped body is produced, is preferably formed to a preponderant extent from gelatin. This includes in particular gelatin fractions of 60% by weight or more, preferably 75% or more. Apart from gelatin, the material may contain for example still further biopolymers such as for example alginates or hyaluronic acid, in order to adapt the profile of characteristics of the shaped bodies more specifically to a particular application.

In order to ensure optimal biocompatibility of the shaped bodies according to the invention in the case of medical use, a gelatin with an especially low content of endotoxins is preferably used as starting material. By endotoxins are meant metabolic products or fragments of microorganisms, which are present in animal raw material. The endotoxin content of gelatin is specified in International Units per gram (I.U./g) and is determined by the LAL test, the carrying out of which is described in the fourth edition of the European Pharmacopoeia (Ph. Eur. 4).

In order to keep the content of endotoxins as low as possible, it is advantageous for the microorganisms to be killed off as early as possible in the course of preparation of the gelatin. Furthermore, suitable standards of hygiene should be observed in the preparation process.

Accordingly, the endotoxin content of the gelatin can be drastically reduced by specific measures during the preparation process. Among these measures, there belong primarily use of fresh raw materials (for example, pig skin) with storage time being avoided, meticulous cleaning of the entire production installation immediately before beginning preparation of the gelatin, and optionally replacement of ion exchangers and filter systems in the production installation.

The gelatin used within the scope of the present invention preferably has an endotoxin content of 1,200 I.U./g or less, still more preferably, 200 I.U./g or less. Optimally, the endotoxin content is 50 I.U./g or less, in each case determined according to the LAL test. By comparison with this, many commercially available gelatins have endotoxin contents of more than 20,000 I.U./g.

According to the invention, in addition to gelatin, the material comprises at least one plasticizer, by which the flexibility of the shaped body is increased and its ability to be stretched is significantly improved. Glycerin, oligoglycerins, oligoglycols and sorbite are for example suitable as plasticizers, glycerin being the most preferred.

The desired flexibility of the shaped body may be controlled by way of the amount of plasticizer. Preferably, the fraction of plasticizer in the material is 12 to 40% by weight. Fractions of 16 to 25% by weight are in this regard especially advantageous.

The stretched shaped body is preferably stretched monoaxially. In this way a preferred direction is defined along which the gelatin molecules are at least in part oriented.

The shaped bodies according to the invention have a high mechanical strength, in particular tear strength. Preferably the shaped bodies according to the invention have a tear strength of 40 N/mm² or more, more preferably 60 N/mm² or more, in each case measured in the direction of stretching.

In addition, the shaped bodies also have, surprisingly, a high ultimate elongation (stretch limit), in particular in the direction of stretch. Preferably the ultimate elongation of the shaped body is then 30% or higher, more preferably 50% or higher, in each case measured in the direction of stretching.

In principle, both the gelatin and also other suitable constituents of the material may be cross-linked in the shaped body. In is however preferred that the gelatin in particular is cross-linked.

The cross-linking may be chemical cross-linking. For this, any cross-linking agent is in principle suitable which effects linking of the individual gelatin molecules with each other. Preferred cross-linking agents are aldehydes, dialdehydes, isocyanates, diisocyanates, carbodiimides and alkyl halides. Especially preferred is formaldehyde, which effects at the same time sterilization of the shaped body.

In a further embodiment of the shaped body according to the invention, the material is cross-linked enzymatically. The enzyme transglutaminase is preferably used as cross-linking agent in this case, transglutaminase effecting linking of glutamine and lysine side chains, in particular also of gelatin.

The shaped bodies according to the invention may have to an extent remarkably long lifespans under physiological conditions, and it is possible to set these lifespans very specifically by the degree of cross-linking. Thus shaped bodies according to the invention may remain stable under standard physiological conditions for example for longer than a week, longer than two weeks or longer than four weeks.

The concept of stability is to be understood to the effect that the shaped body substantially retains its original shape both during storage in the dry state and also during the specified time period under standard physiological conditions and only subsequently breaks down structurally to a significant extent by hydrolytic action.

Physiological conditions to which the shaped bodies are exposed when used to produce implants are primarily characterized by temperature, pH value and ion strength. Corresponding conditions may be simulated in vitro by incubation in PBS buffer (pH 7.2, 37° C.), in order to test and compare different shaped bodies in respect of their time-dependent stability properties (called standard physiological conditions in the following text).

The mechanical strength of the shaped bodies according to the invention may be increased by the addition of a reinforcing material. The reinforcing material should be physiologically compatible and at best also resorbable.

Depending on the choice of reinforcing material, the stability of the shaped body in respect of resorption mechanisms may be affected to a certain extent, along with the effect on mechanical properties. In particular, the resorption stability of the reinforcing materials may be selected independently of the constituents of the gelatinous material.

The reinforcing materials show, even for fractions of 5% by weight (relative to the total mass of the shaped body), a marked improvement in the mechanical properties of the shaped body.

Above 60% by weight, no further significant improvement can as a rule be achieved and/or the desired resorption properties or also the necessary flexibility of the shaped body may be achieved only with difficulty.

Reinforcing materials may be selected from particulate and/or molecular reinforcing materials as well as mixtures of these.

In the case of particulate reinforcing materials, the use of reinforcing fibers is particularly recommended. The fibers for this are selected preferably from polysaccharide fibers and protein fibers, in particular collagen fibers, silk and cotton fibers, and from polyactide fibers and mixtures of any of the foregoing.

On the other hand, molecular reinforcing materials are also suitable in order to improve mechanical properties and, if desired, also to improve the resorption stability of the shaped body.

Preferred molecular reinforcing materials are in particular polyactide polymers and their derivatives, cellulose derivatives, and chitosan and its derivatives. Molecular reinforcing materials may also be used as mixtures.

In a preferred embodiment of the shaped body according to the invention, the body is a film. Films of this kind based on a cross-linked, gelatinous material may be used in a diversity of ways to cover over and/or protect damaged tissue, for population with cells and for production of combination materials in conjunction with shaped bodies having a cell structure, for example sponges.

The thickness of the films according to the invention is preferably 20 to 500 μm, most preferably 50 to 250 μm.

A further preferred embodiment of the shaped body according to the invention relates to a hollow cylinder. Hollow cylinders of this kind may be used inter alia as nerve guides. In this regard, implants are in question which allow regeneration of severed nerve members, in that in each case an individual nerve cell grows along the cavity of the nerve guide.

Hollow cylinders according to the invention may be stretched both in the longitudinal direction and in the circumferential direction. The actual production of a hollow cylinder of this kind is gone into in detail later on below.

In the case of hollow cylinders which are stretched in the longitudinal direction, not only their mechanical properties are improved by stretching but at the same time, hollow cylinders are provided which have a smaller internal diameter compared with unstretched hollow cylinders. The internal diameter can thereby be adapted to the respective requirements, for example to the dimensions of the nerve cells in the case of the hollow cylinders being used as nerve guides.

Depending on the use, the hollow cylinder may have an internal diameter of 300 to 1,500 μm, preferably 900 to 1,200 μm. The average wall thickness of the hollow cylinder is preferably in the range from 140 to 250 μm.

It is a further object of the present invention to provide a method by which there may be produced shaped bodies based on gelatin, which have improved mechanical properties.

This object is met according to the invention by a method which comprises the following steps:
a) preparing an aqueous solution of a gelatinous material;
b) partially cross-linking the dissolved, gelatinous material;
c) producing a shaped body starting from the solution containing the partially cross-linked material; and
d) stretching the shaped body.

As has already been set out in connection with the shaped bodies according to the invention, the mechanical strength of the shaped bodies may be significantly increased by stretching. According to the invention, the stretching for this is effected after the gelatinous material has been partially cross-linked. This sequence leads to better results than stretching the shaped body before the cross-linking as per the prior art (Bigi at al. (1998) Biomaterials 19, 2335-2340; see above).

The gelatinous material used in step a) is preferably formed to a preponderant extent from gelatin. This includes in particular gelatin fractions of 60% by weight or more, preferably 75% or more. In addition, the material, as described above, may contain further constituents.

In principle, gelatins of different origin and quality may be used as starting material for the method; in respect of medical usage, the use of gelatins which are low in endotoxins is however preferred, as described above. The gelatin concentration in the solution in step a) may for this be 5 to 45% by weight, preferably 10 to 30%.

The material in step a) preferably comprises in addition a plasticizer. The stretchability of the shaped body is substantially improved by this, as has already been described in connection with the shaped bodies according to the invention.

Suitable plasticizers are for example glycerin, oligoglycerins, oligoglycols and sorbite, glycerin being most preferred. Advantageously, the fraction of plasticizer in the material is 12 to 40% by weight. Most preferred for this are fractions from 16 to 25% by weight.

The shaped body formed in step c) is preferably at least partially dried before stretching (step d)), preferably to a residual moisture content of less than 20% by weight, in particular 15% by weight or less.

Preferably the shaped body is brought into a thermoplastic state directly before the stretching (step d)), by raising temperature and/or water content. This may for example be accomplished by the shaped body being exposed to hot steam. Stretching of the shaped bodies is advantageously carried out with a stretch ratio of 1.4 to 8, a stretch ratio of up to 4 being preferred.

In a particular embodiment of the method according to the invention, step d) is carried out up to 4 weeks after step c). By storing the shaped body prior to stretching, the storage being preferably at room temperature, the strength of the shaped bodies produced according to the invention can to an extent be significantly increased. For this, step d) is preferably carried out three to seven days after step c).

A further embodiment of the method according to the invention comprises a further step e), in which the material comprised in the stretched shaped body undergoes additional cross-linking.

The gelatin and/or another suitable constituent of the material may be cross-linked both in step b) and also in the optional step e). Preferably, the gelatin in particular is cross-linked in both cases.

The advantage of two-stage cross-linking resides principally in its being possible to achieve a higher degree of cross-linking and thereby, as a result, extended times to degradation. This cannot be realised to the same extent by a single-stage method in which the concentration of cross-linking agent is increased, because the dissolved material can no longer be worked and brought into a shape if has been cross-linked to too great an extent.

On the other hand, cross-linking of the material exclusively after production of the shaped body is also unsuitable, since in this case, the boundary surfaces accessible from the outside are more strongly cross-linked than in the inner regions of the shaped body, which is reflected in non-homogeneous breakdown behavior.

Stretching according to the invention of the shaped body between the two cross-linking steps is especially advantageous because the molecules in the partially cross-linked material still have sufficient freedom of movement and can therefore be oriented at least partially along a preferred direction.

The second cross-linking (step e)) may be carried out by the action of an aqueous solution of a cross-linking agent, but is however preferably effected by a gaseous cross-linking agent.

In step b) and optional step e), the same or different cross-linking agents may be used, preferred chemical and enzymatic cross-linking agents having already been described in connection with the shaped bodies according to the invention. Formaldehyde is especially preferred, in particular for the optional second cross-linking step in the gas phase, since the shaped body may at the same time be sterilised by formaldehyde. In this way, the action of the formaldehyde on the shaped body may be effected, supported by a steam atmosphere.

The cross-linking agent in step b) is preferably added to the solution in an amount of 600 to 5,500 ppm, preferably 2,000 to 4,000 ppm, relative to the gelatin.

By varying the concentration of cross-linking agent in the solution, but also by different levels of cross-linking in the second cross-linking step, both the mechanical strength of the shaped bodies produced and their lifespan under physiological conditions may be set in a very simple way. Thus, surprisingly, shaped bodies may be obtained, which on the one hand remain stable under physiological conditions for example for longer than a week, longer than two weeks or longer than four weeks and on the other hand, satisfy demands in respect of cell compatibility and resorbability.

In a particular embodiment of the method according to the invention, the shaped body is a film. Films may in particular be produced by casting or extrusion of the solution in step c).

In another embodiment of the method according to the invention, the shaped body is a hollow cylinder. Hollow cylinders may also be produced by extrusion of the solution in step c). Preferred however is production of hollow cylinders by uniform application of the solution in step c to the surface of a cylinder, in particular by briefly dipping the cylinder into the solution. When the solution dries, there results a hollow cylinder which can be pulled off the cylinder.

A further preferred production method for hollow cylinders comprises rolling a film up to form a single-layer or multi-layer hollow cylinder. Bonding of the film to form a closed hollow cylinder may for example be effected by the film being moist during the rolling up, and being thereby adhered. Alternatively, the film may be bonded by an adhesive, for example gelatin.

In one embodiment of the method, the hollow cylinder is initially formed by rolling up an unstretched film, (steps a) to c)) and is then stretched in the longitudinal direction (step d)), the internal diameter being thereby reduced (see above). The hollow cylinder produced by dipping may also be stretched in this way.

In an alternative embodiment of the method, a film is first of all produced and stretched (steps a) to d) and only after that is it rolled up to form a hollow cylinder. The rolling up can then be effected either parallel to the direction of stretching or at right angles to it, hollow cylinders with increased tear strength in the longitudinal direction or in the circumferential direction being obtained. Depending on the field of use, the one or the other variant may be preferred.

Rolling up films at right angles to the direction of stretching is especially advantageous for fiber-reinforced films, since in this case the fibers are oriented at least in part along the circumferential direction of the hollow cylinder. For use as nerve guides, which are often surgically stitched at their ends, fiber orientation of this kind can resist any tearing-out of the threads of the stitches.

The method according to the invention is particularly suitable for production of the shaped bodies according to the invention, described above. Further advantages of the production method are thus also apparent from the description of the shaped bodies according to the invention.

The invention further relates to use of the shaped bodies described for use in the fields of human and veterinary medicine and for producing implants.

One use according to the invention relates in one aspect to the production of covers for wounds from the shaped bodies previously described. These may be used for treating wounds or internal or external bleeding, for example during operations. Resorption of the shaped body is then effected after an individually determinable time, preferably by selection of production conditions.

It has been shown that shaped bodies according to the invention are eminently suitable for population with mammalian cells, i.e. human or animal cells. For this, a shaped body is treated with a suitable nutrient solution and the cells, for example fibroblasts or chondrocytes, are then seeded-out onto it. Because of the stability of the material, the cells can grow and proliferate in vitro for several weeks.

The invention further relates to implants, in particular tissue implants, which comprise a shaped body according to the invention, and cells applied to this or cultivated on it, as described above.

Implants according to the invention are used for treatment of tissue defects, for example skin or cartilage defects, the seeded-out cells being for example taken previously from the patient. During the growth phase of the cells, the shaped body protects the tissue forming from mechanical stress, and the formation of the cells' own extracellular matrix is enabled. Both the high mechanical strength and the adjustable resorption time of the shaped body according to the invention prove to be of especial advantage for this. By means of long life materials, which have a resorption time of more than four weeks, either large-scale defects or defects in tissue types with slow cell growth may be treated.

Finally, the invention relates to a nerve guide comprising a shaped body according to the invention, in the form of a hollow cylinder. Particular advantages and embodiments of nerve guides of this kind have already been described extensively above.

### BRIEF DESCRIPTION OF THE SEVERAL VIEWS OF THE DRAWINS

These and further advantages of the invention will be explained in more detail on the basis of the accompanying examples with reference to the figures. In particular:

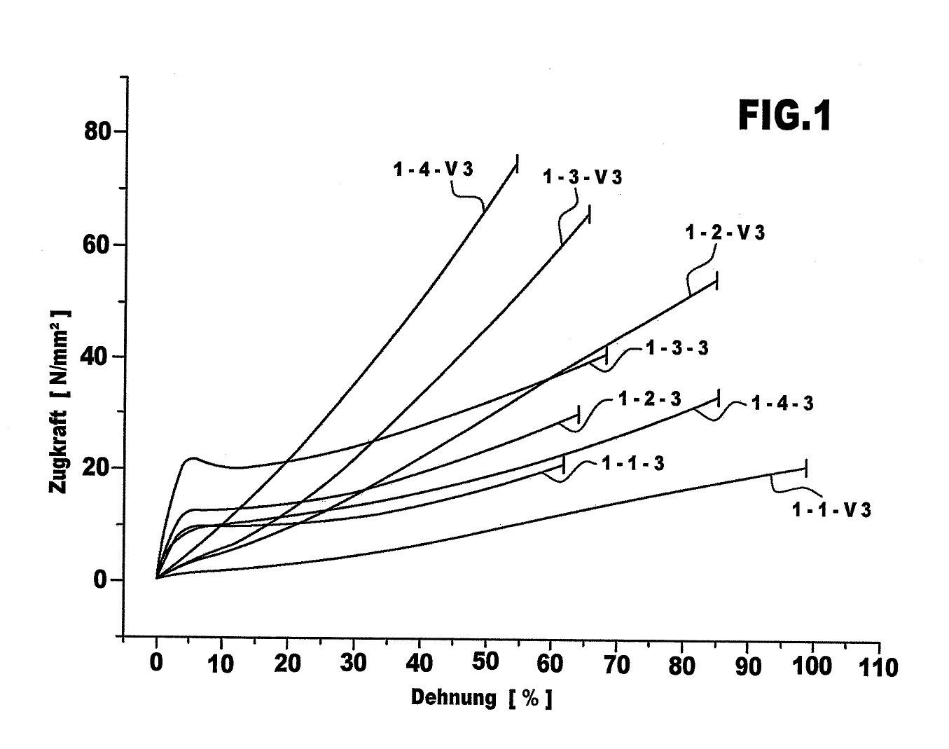

FIG. 1: shows a strain/elongation diagram for shaped bodies according to the invention in the form of films which have different degrees of cross-linking, having been stretched after a storage time of three days;

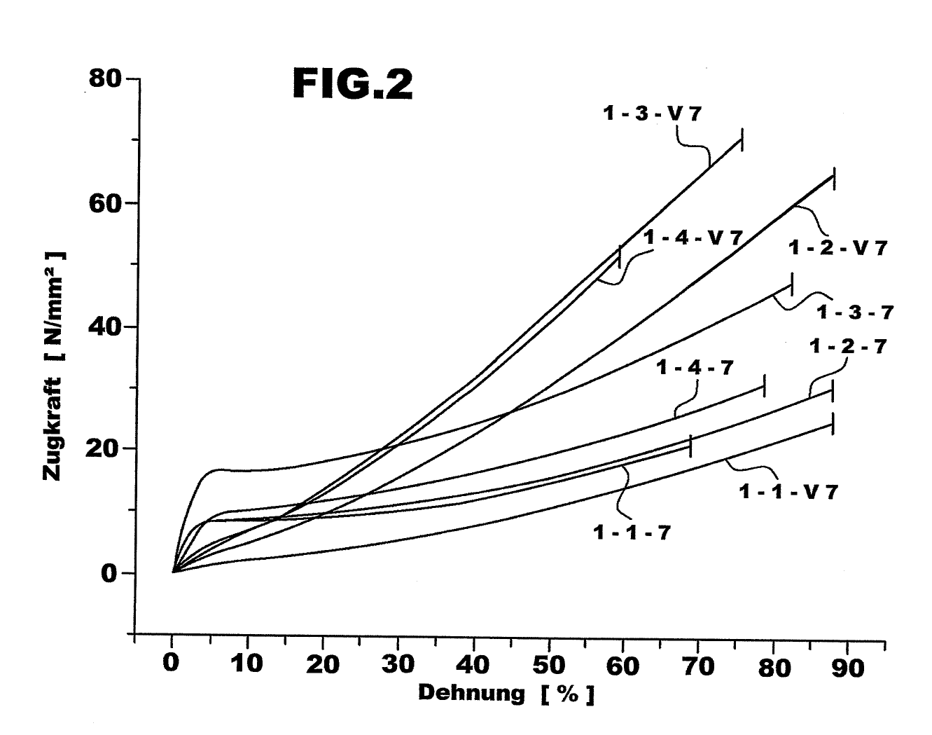

FIG. 2: shows a strain/elongation diagram for shaped bodies according to the invention in the form of films which have different degrees of cross-linking, having been stretched after a storage time of seven days;

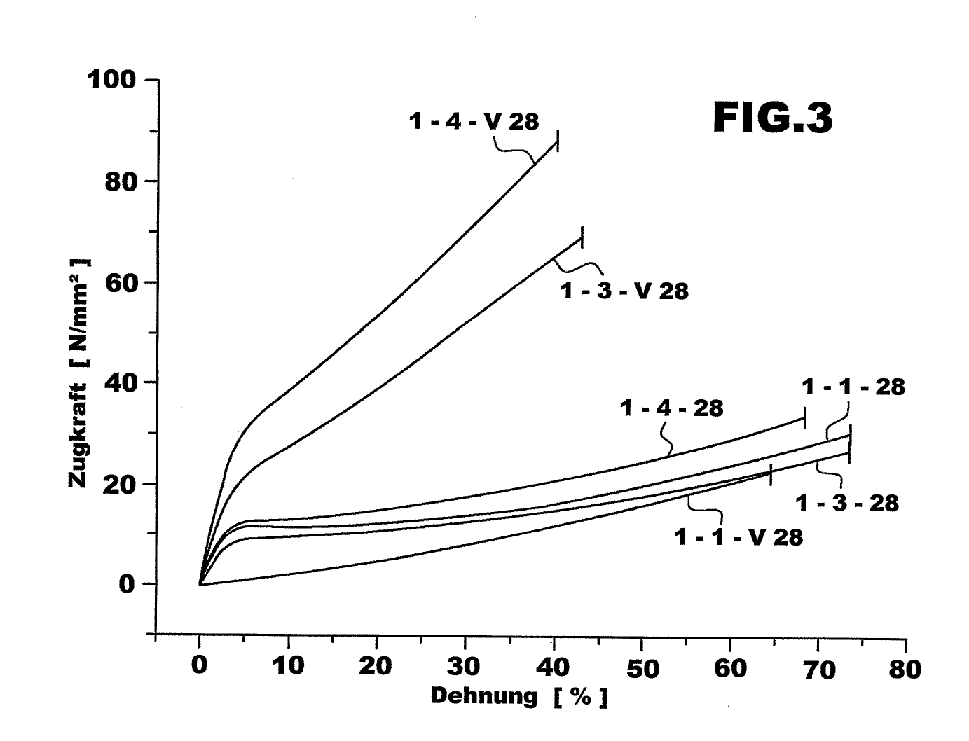

FIG. 3: shows a strain/elongation diagram for shaped bodies according to the invention in the form of films which have different degrees of cross-linking, having been stretched after a storage time of 28 days;

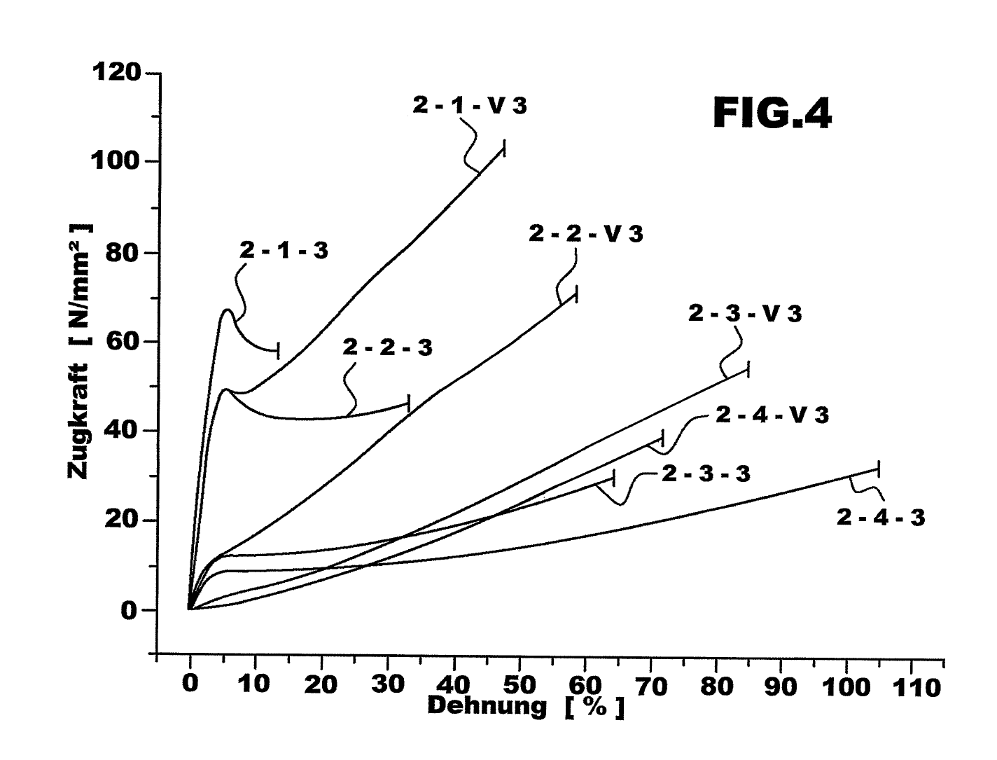

FIG. 4: shows a strain/elongation diagram for shaped bodies according to the invention in the form of films which have a different fraction of plasticizer, having been stretched after a storage time of three days;

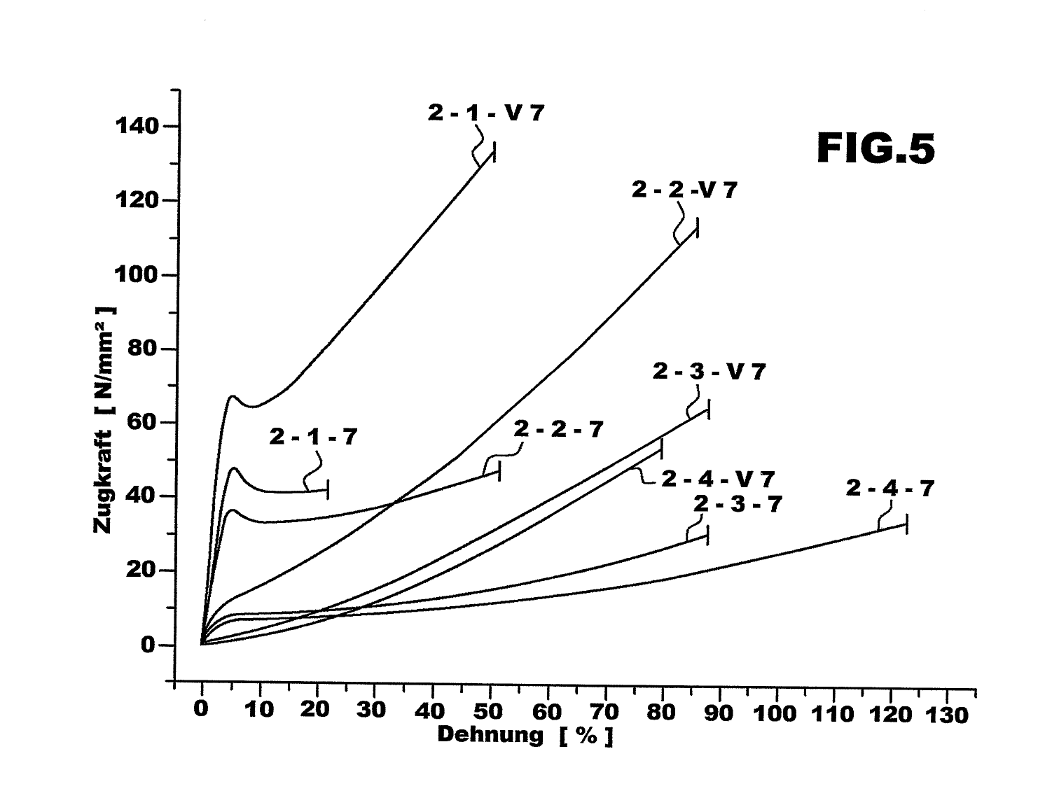

FIG. 5: shows a strain/elongation diagram for shaped bodies according to the invention in the form of films which have a different fraction of plasticizer, having been stretched after a storage time of seven days;

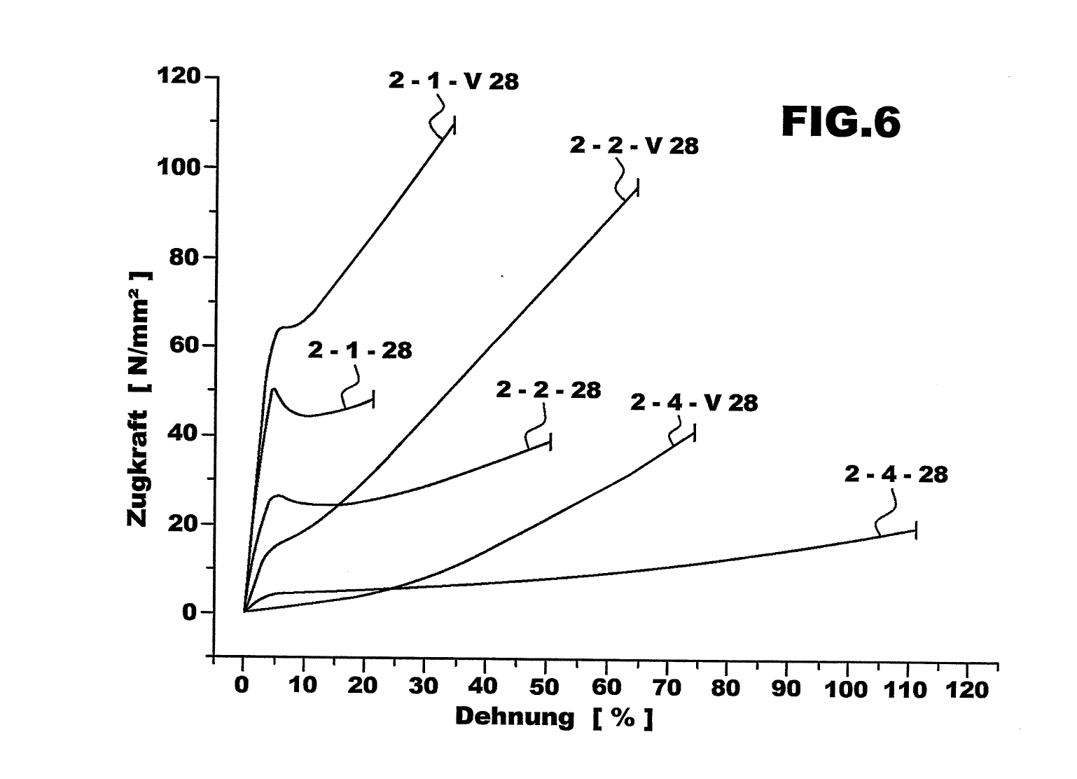

FIG. 6: shows a strain/elongation diagram for shaped bodies according to the invention in the form of films which have a different fraction of plasticizer, having been stretched after a storage time of 28 days;

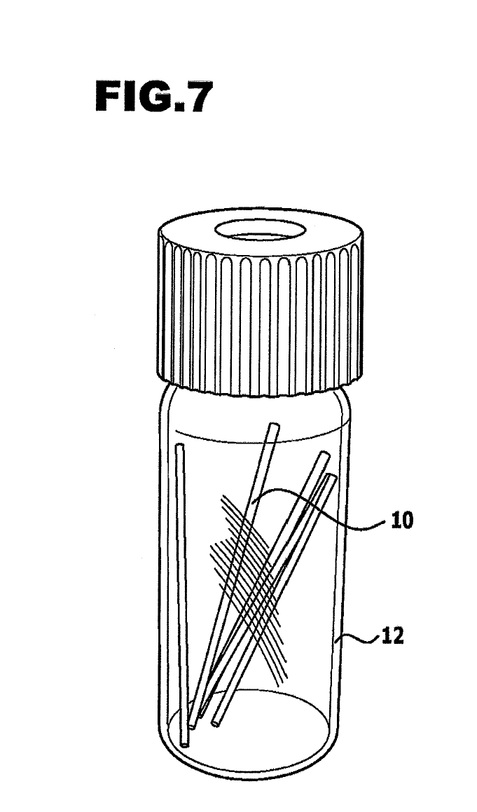

FIG. 7: shows a photographic illustration of a hollow cylinder according to the invention; and

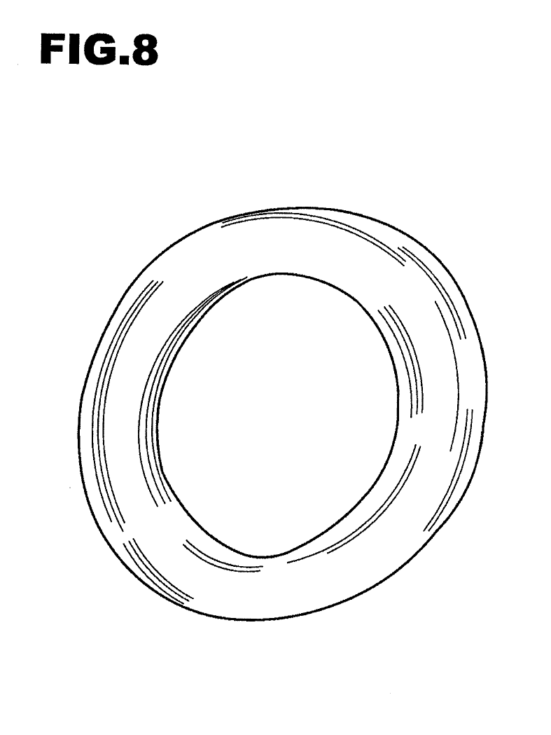

FIG. 8: shows an image, taken using an optical microscope, of a hollow cylinder according to the invention, in cross-section.

### EXAMPLES

### Example 1

### Production and Properties of Stretched and Unstretched Films which Have Different Degrees of Cross-Linking

For this example, different films were produced based on a material which in each case contained constant fractions of about 71% gelatin by weight and about 29% plasticizer by weight. The different quantities of cross-linking agent were between 1,000 and 4,000 ppm (in each case with reference to the quantity of gelatin).

For this, 20 g of pig skin gelatin with a Bloom strength of 300 g was, for each formulation, dissolved at 60° C. in a mixture of 72 g of water and 8 g of glycerin as plasticizer. After the solutions were degassed by means of ultrasound, the quantities indicated in Table 1 of an aqueous formaldehyde solution (2.0% by weight, room temperature) were in each case added, the mixture was homogenized, and squeegeed out at about 60° C. to a thickness of 1 mm on a polyethylene underlay.

TABLE 1

| | Formulation | | | | | | | |
|---|---|---|---|---|---|---|---|---|
| | 1-1 | | 1-2 | | 1-3 | | 1-4 | |
| Formaldehyde | 1 | g | 2 | g | 3 | g | 4 | g |
| solution | | | | | | | | |
| Content of | 1,000 | ppm | 2,000 | ppm | 3,000 | ppm | 4,000 | ppm |
| formaldehyde | | | | | | | | |
| with reference | | | | | | | | |
| to gelatin | | | | | | | | |

After drying at 25° C. and a relative humidity of 30% for about two days, the films produced were peeled off from the PE-underlay. The thickness of the films was about 220 μm.

Before stretching, different films produced in accordance with the above formulations 1-1 to 1-4 were stored for three, seven and 28 days respectively at a temperature of 23° C. and a relative humidity of 45%. Corresponding films which were not stretched were in each case treated in the same way.

For stretching, the films were softened by the action of hot steam, elongated in this thermoplastic state up to the stretch limit and fixed overnight at a temperature of 23° C. and a relative humidity of 45%. The stretch ratio was thereby in a range from 2 to 4.

The strain/elongation diagram for the stretched films (in the direction of stretch) as well as that for the corresponding unstretched films was then plotted. These are shown in FIGS. 1 to 3.

In the labelling of the individual curves in the diagrams, the first two digits represent in each case the formulation from which the film was produced, while the third digit represents the time for which the film was stored before stretching (three, seven or 28 days). Stretched films are designated by the letters V before the final digit.

FIG. 1 shows the strain/elongation diagram for the films stretched after three days as well as that for the unstretched films which had been stored for three days under the same conditions. Comparison of the curves with one another shows first of all that the tear strength of the films stretched according to the invention increases significantly with increase in the content of cross-linking agent.

The effects of stretching are also dependent on the content of cross-linking agent. For the relatively low formaldehyde content of 1,000 ppm, the tear strength of the stretched film 1-1-V3 remains largely constant as compared with the unstretched film 1-1-3, while the ultimate elongation is raised significantly from about 60% to almost 100%. For formaldehyde concentrations of 2,000 ppm and more, stretching leads to films which have a significantly raised tear strength, in the case of a formaldehyde content of 4,000 ppm, this being even more than doubled (film 1-4-V3 compared with film 1-4-3).

These results show that by stretching films based on cross-linked gelatin, the mechanical properties of the films may be improved in very many ways. Depending on the degree of cross-linking, there results a positive effect on the ultimate elongation, the tear strength, and also on both parameters at the same time (for example film 1-2-3 compared with film 1-2-3).

FIG. 2 shows the strain/elongation diagram for the films stretched after seven days as well as that for the unstretched films. The higher tear strength for the films achieved by stretching is also clearly apparent here.

Comparison with FIG. 1 also shows that by virtue of the longer storage time before stretching, higher tear strengths may be achieved for the films according to the invention, even at lower contents of cross-linker (for example, film 1-2-V7 compared with film 1-2-V3). The cause of this is probably continuation of the cross-linking reaction during the storage period.

Finally, FIG. 3 shows the mechanical properties for the films stretched after 28 days, along with those for the corresponding reference films. The strain/elongation diagrams are plotted here only for the films in accordance with the formulations 1-1, 1-3 and 1-4.

While the curves for the unstretched films are almost identical after a storage time of 28 days, the properties of the stretched films are to a great extent dependent on the content of cross-linking agent. For a low content of 1,000 ppm, stretching has hardly any effect, but for 3,000 and 4,000 ppm, by contrast, the tear strength increases dramatically as compared with the unstretched films. The maximum tear strength of almost 90 N/mm², which is achieved for the film 1-4-V28, is, by virtue of the long storage time, higher still than in the case of the films stretched after three or seven days.

For all of the strain/elongation diagrams illustrated, it must be taken into account that the respective curves are not precisely reproducible in the production of films under laboratory conditions. The relationship of the curves of different films to one another is however typical.

### Example 2

### Production and Properties of Stretched and Unstretched Films Which Have Different Fractions of Plasticizer

This example relates to films based on cross-linked gelatin which has a constant content of cross-linking agent of 2,000 ppm (with reference to the quantity of gelatin). As well as gelatin, the material for the films also comprised different fractions of plasticizer, between about 17% by weight and about 33% by weight.

For producing the films, 20 g of pig skin gelatin (Bloom strength 300 g) were in each case dissolved at 60° C. in a mixture of water and glycerin as plasticizer, in four different formulations, respectively according to the quantities given in Table 2. After the solution was degassed by means of ultrasound, 2 g of an aqueous formaldehyde solution (2.0% by weight, room temperature) were in each case added, the mixture was homogenized, and squeegeed out at about 60° C. to a thickness of 1 mm on a polyethylene underlay.

TABLE 2

| | Formulation | | | | | | | |
|---|---|---|---|---|---|---|---|---|
| | 2-1 | | 2-2 | | 2-3 | | 2-4 | |
| Water | 76 | g | 74 | g | 72 | g | 70 | g |
| Glycerin | 4 | g | 6 | g | 8 | g | 10 | g |
| Fraction of | 16.7% | by | 23.1% | by | 28.6% | by | 33.3% | by |
| glycerin in the | | weight | | weight | | weight | | weight |
| material | | | | | | | | |

The drying, storage and stretching of the films were also effected in this case as described in Example 1.

The strain/elongation diagrams for the stretched and unstretched films are shown in FIGS. 4 to 6. The designations of the individual curves are analogous to Example 1.

FIG. 4 shows the strain/elongation diagram for the films according to the invention which were stretched after a storage time of three days as well as that for the corresponding unstretched films. The first matter to draw attention is that for all of the fractions of plasticizer used, the tear strength of the films according to the invention is significantly increased by stretching. This effect is especially striking for the films of formulations 2-1 and 2-2 which have a low fraction of plasticizer and have, in the absence of stretching, an entirely unsatisfactory strain/elongation relationship. The stretched films have by contrast very good mechanical properties with high tear strengths (about 100 N/mm² for the film 2-1-V3).

It is further to be noted that stretching, in accordance with the invention, of the films significantly improves not only the tear strength but, with the exception of formulation 2-4, also the ultimate elongation of the films. This is most surprising when it is considered that the films have already experienced an elongation of about 100 to 300% during stretching.

The strain/elongation diagram for the films stretched after seven days show the same results qualitatively as those for stretching after three days. For all formulations, the tear strength of the films stretched according to the invention are in part significantly higher by virtue of the longer storage time, which may be ascribed primarily to the above-described continuation of the cross-linking reaction. The longer storage also has a positive influence on the ultimate elongations.

Finally, FIG. 6 shows the strain/elongation diagrams for the films in the case of a storage time of 28 days, here only the stretched and unstretched films for the formulations 2-1, 2-2 and 2-4 being measured. Compared with FIG. 5, the curves run very similarly, the tear strengths of the stretched films being in fact somewhat lower again than for the seven-day storage. This suggests that there is an optimum for the storage time, which may be dependent on the concentration of the cross-linking agent and the fraction of plasticizer.

### Example 3

### Production of Stretched Films Which Have Been Cross-Linked Twice

This example relates to the production of films according to the invention comprising a second cross-linking step after stretching, by virtue of which the times for physiological degradation of the films are significantly increased.

The starting point for this was the stretched films of Examples 1 and 2. After they had been stretched and fixed overnight, these were exposed, in a dessicator, for two hours to the equilibrium vapor pressure of an aqueous formaldehyde solution of 17% by weight, at room temperature.

The breakdown properties of these twice cross-linked films were then studied in respect of their difference from the starting films which had been cross-linked once. For this, film portions of 2×3 cm² size where placed in each case in a 500 ml PBS-buffer (pH 7.2) and the concentration of the gelatin dissolved in the buffer measured at a wavelength of 214 nm. While the films that had been cross-linked once were fully dissolved after 15 minutes, no change was discerned for the twice cross-linked films even after an hour.

The advantageous mechanical properties of the stretched films remain substantially unaffected by the second cross-linking step.

### Example 4

### Production of Enzymatically Cross-Linked Films Based on Gelatin

This example relates to the production of a film based on gelatin, the cross-linking being carried out enzymatically by transglutaminase.

For this, 20 g of pig skin gelatin (Bloom strength 300 g) was dissolved at 60° C. in a mixture of 72 g of water and 8 g of glycerin, which equated to a fraction of plasticizer of about 29%. After the solution was degassed by means of ultrasound, 4 g of an aqueous transglutaminase solution with a specific activity of 30 U/g were added, the mixture was homogenized, and squeegeed out to a thickness of 1 mm on a polyethylene underlay heated to 45° C.

After 30 minutes, the film was peeled off from the PE-underlay, was held for 2 hours at a temperature of 50° C. and a relative humidity of 90% and then dried for about two days at a temperature of 25° C. and a relative humidity of 30%.

The film cross-linked using transglutaminase exhibited a tear strength of about 9 N/mm² for an ultimate elongation of about 300%.

Stretching of the film produced in this way and possibly a second cross-linking using formaldehyde in the gas phase may be carried out in the same way as is described in Examples 1 to 3.

### Example 5

### Production of Stretched Hollow Cylinders Based on Gelatin

By stretching according to the invention of hollow cylinders based on gelatin, very thin tubules may be produced which have an internal diameter in the range from 800 to 1,200 μm.

A solution of pig skin gelatin (Bloom strength 300 g) serves as starting material, which, corresponding to the procedure described in Examples 1 and 2, was prepared by dissolving 100 g of gelatin in a mixture of 260 g of water and 40 g of glycerin as plasticizer. This equated to a fraction of plasticizer of about 29% by weight.

After addition of 4 g of an aqueous formaldehyde solution of 2.0% by weight (800 ppm of cross-linker relative to the gelatin), the solution was homogenised, once again degassed and the surface freed from foam. An array of stainless steel pins with a diameter of 2 mm, which had previously been sprayed with a separating wax, was dipped briefly into the solution to a length of about 3 cm. After the pins were withdrawn from the solution, they were held vertical, so that the solution adhering formed as uniform a layer as possible.

After drying for approximately one day at 25° C. and a relative humidity of 30%, it was possible to remove the formed gelatin tubules from the stainless steel pins. These were then stored for a further five days at 23° C. and a relative humidity of 45%.

For stretching, the tubules were gripped at both ends and softened by the action of hot steam. In this thermoplastic condition, they were lengthened with a stretch ratio of about 1.4, fixed in this condition, and dried overnight at 23° C. and a relative humidity of 45%.

In order to prolong the time for physiological degradation of the tubules, they were submitted to a second cross-linking step, corresponding to the films described in Example 3. For this, the tubules were exposed, in a dessicator, for 17 hours to the equilibrium vapor pressure of an aqueous formaldehyde solution of 17% by weight, at room temperature. During this, the ends of the tubules were closed, so that the cross-linking was effected only from the outside inward.

In FIG. 7, some of the gelatin tubules 10 produced in this way and having a length of about 3 cm, are shown in a glass container 12.

FIG. 8 shows an image taken using an optical microscope of the cross-section through one of the tubules. The tubule depicted has an internal diameter of about 1,100 μm and a wall thickness of about 200 μm: both the cross-sectional shape and the wall thickness of the tubule are extremely consistent.

The gelatin tubules produced in this example are especially well suited for use as nerve guides on account their dimensions and on account of the long time they require for degradation. Also, the stronger cross-linking of the tubule starting from the outer side is advantageous for this use, since in this way, the tubule can become broken down starting from the inside outward as the nerve cell grows.

By raising the stretch ratio, hollow cylinders according to the invention with an even smaller internal diameter may also be produced, which may be advantageous for other uses. In particular, it is possible by use of the method according to the invention, to produce extremely thin tubules having an internal diameter in the region of 150 μm. A value of this level cannot be achieved other than by stretching the tubule.

1. A shaped body comprising a cross-linked, gelatinous material which comprises gelatin and a plasticizer, the shaped body being stretched so that the gelatin molecules are oriented at least in part in a preferred direction.

2. The shaped body according to claim 1, the material being formed to a preponderant extent from gelatin.

3. The shaped body according to claim 1, the gelatin having an endotoxin content, as determined by the LAL test, of 1,200 I.U./g or less.

4. The shaped body according to claim 1, the plasticizer being selected from glycerin, oligoglycerins, oligoglycols and sorbite.

5. The shaped body according to claim 1, the fraction of plasticizer in the material being 12 to 40% by weight.

6. (canceled)

7. The shaped body according to claim 1, the shaped body being stretched monoaxially.

8. The shaped body according to claim 1, the shaped body having an ultimate elongation, measured in the direction of stretching, of 30% or higher.

9. The shaped body according to claim 8, the shaped body having an ultimate elongation, measured in the direction of stretching, of 50% or higher.

10. the shaped body according to claim 1, the shaped body having a tear strength, measured in the direction of stretching, of 40 N/mm² or higher.

11. The shaped body according to claim 10, the shaped body having a tear strength, measured in the direction of stretching, of 60 N/mm² or higher.

12-19. (canceled)

20. The shaped body according to claim 1, further comprising a reinforcing material.

21. The shaped body according to claim 20, the reinforcing material being present in the shaped body in a fraction of 5% by weight or more.

22. The shaped body according to claim 20, the reinforcing material being present in the shaped body in a fraction of up to 60% by weight.

23. The shaped body according to claim 20, the reinforcing material being selected from particulate and/or molecular reinforcing materials.

24. The shaped body according to claim 23, the particulate reinforcing material comprising reinforcing fibers.

25. The shaped body according to claim 24, the reinforcing fibers being selected from polysaccharide fibers and protein fibers and from polyactide fibers and mixtures of any of the foregoing.

26. The shaped body according to claim 23, the molecular reinforcing material being selected from polyactide polymers and their derivatives, cellulose derivatives and chitosan and its derivatives.

27. The shaped body according to claim 1, the shaped body being a film.

28. The shaped body according to claim 27, the film having a thickness of 20 to 500 μm.

29. The shaped body according to 1, the shaped body being a hollow cylinder.

30. the shaped body according to claim 29, the hollow cylinder being stretched in the longitudinal direction.

31. The shaped body according to claim 29, the hollow cylinder being stretched in the circumferential direction.

32. The shaped body according to claim 29, the hollow cylinder having an internal diameter of 300 to 1,500 μm.

33. The shaped body according to claim 29, the hollow cylinder having an average wall thickness of 140 to 250 μm.

34. A method for producing a stretched shaped body comprising a cross-linked, gelatinous material, the method comprising:
a) preparing an aqueous solution of a gelatinous material;
b) partially cross-linking the gelatinous material in the solution;
c) producing a shaped body starting from the solution containing the partially cross-linked material; and
d) stretching the shaped body.

35. (canceled)

36. The method according to claim 34, wherein preparing the aqueous solution of the gelatinous material comprises adding a plasticizer to the material.

37-39. (canceled)

40. The method according to claim 34, comprising at least partially drying the shaped body between c) and d).

41. The method according to claim 34, comprising bringing the shaped body into a thermoplastic state directly before d), by raising temperature and/or water content.

42. The method according to claim 34, wherein stretching the shaped body comprises stretching the shaped body with a stretch ratio of 1.4 to 8.

43. (canceled)

44. (canceled)

45. The method according to claim 34, wherein stretching the shaped body is carried out three to seven days after producing the shaped body according to c).

46. (canceled)

47. The method according to claim 34, further comprising: e) further cross-linking the material comprised in the stretched shaped body.

48. (canceled)

49. The method according to claim 47, wherein further cross-linking of the gelatinous material comprises exposing the stretched shaped body to a cross-linking agent in the gas phase.

50. (canceled)

51. (canceled)

52. The method according to claim 34, wherein the partially crosslinking gelatinous material according to b) comprises adding a cross-linking agent to the solution in an amount of 600 to 5,500 ppm, relative to the gelatin.

53. (canceled)

54. (canceled)

55. The method according to claim 34, the shaped body being a film.

56. The method according to claim 55, wherein producing the shaped body according to c) comprising casting or extrusion of the solution.

57. The method according to any of claim 34, the shaped body being a hollow cylinder.

58. The method according to claim 57, wherein producing the shaped body according to c) comprises application of the solution to the surface of a cylinder.

59. The method according to claim 57, the method further comprising rolling up a film produced in accordance with c) to form a single-layer or multi-layer hollow cylinder.

60. The method according to claim 59, comprising rolling up the film before stretching.

61. The method according to claim 59, comprising rolling up the film after stretching.

62. The method according to claim 61, comprising rolling up the film parallel to the direction of stretching.

63. The method according to claim 61, comprising rolling up the film at right angles to the direction of stretching.

64-68. (canceled)

69. An implant comprising a shaped body according to claim 1 and mammalian cells in or on the shaped body.

70-72. (canceled)

73. The shaped body according to claim 3, the gelatin having an endotoxin content, as determined by the LAL test, of 200 I.U./g or less.

74. The implant according to claim 69, the mammalian cells being fibroblasts or chrondrocytes.
